(19) United States
(12) Patent Application Publication
(10) Pub. No.: US 2002/0071871 A1
(43) Pub. Date: Jun. 13, 2002

(54) Apparatus and process to produce particles having a narrow size distribution and particles made thereby

(76) Inventors: Herm Snyder, Belmont, CA (US); Adrian E. Smith, Belmont, CA (US); Jim Nasiatka, San Francisco, CA (US)

Correspondence Address: INHALE THERAPEUTIC SYSTEMS, INC, 150 INDUSTRIAL ROAD, SAN CARLOS, CA, 94070, US

(21) Appl. No.: 09/919,278

(22) Filed: Jul. 31, 2001

Related U.S. Application Data
(60) Provisional application No. 60/222,067, filed on Aug. 1, 2000.

(51) Int. Cl. A61K 9/14; A61L 9/04; B29B 9/00

(52) U.S. Cl. 424/489; 424/46; 264/12

(57) ABSTRACT

The present invention is directed to particles, including liquid droplets and dry particulates, having a narrow particle size distribution made from a liquid feed stock. In particular, the invention is directed to producing particles of a desired median diameter and narrow particle size distribution without the need for additional separation processing. The process of the present invention can be tailored to produce substantially monodisperse particles or multimodal particles having well defined and controllable particle size distributions. The present invention is particularly well suited for producing particles for pulmonary administration.

### RELATED APPLICATIONS

The present application claims the benefit of U.S. Provisional Application No. 60/222,067 filed on Aug. 1, 2000.

### FIELD OF THE INVENTION

The present invention is directed to particles having a narrow particle size distribution made from a liquid feed stock. In particular, the invention is directed to producing particles of a desired median diameter and narrow particle size distribution without the need for additional separation processing. The process of the present invention can be tailored to produce substantially monodisperse particles or multimodal particles having well defined and controllable particle size distributions. The present invention is particularly well suited for producing particles for pulmonary administration.

### BACKGROUND OF THE INVENTION

The ability to accurately and reproducibly produce particles having a well defined particle size and particle size distribution from a liquid feed stock has application in a variety of fields, including food, chemicals, and pharmaceuticals. Such control of particle size and particle size distribution is perhaps most critical in pharmaceutical applications such as pulmonary drug delivery where liquid or dry powder particles containing an active agent are administered to a patient. Control over particle size and particle size distribution is necessary in such applications in order to achieve delivery of such particles to the deep lung.

Powders for pulmonary drug administration have been made by spray drying. Spray drying is a conventional chemical processing unit operation used to produce dry particulate solids from a variety of liquid and slurry materials. The process involves rapidly transforming a liquid feed into a dried particulate form by atomizing the feed into a hot drying medium. It is a common method for preparing solids in the chemical, food, and pharmaceutical industries.

The application of conventional spray drying technology to the field of pulmonary drug administration presents many technical challenges which must be overcome. For example, many pharmaceutical agents, including peptides and proteins, are sensitive to thermal degradation and other rigorous treatment conditions. The use of spray drying for the preparation of pharmaceutical formulations, including proteins and polypeptides, can be problematic since such pharmaceutical agents are often labile and subject to degradation when exposed to high temperatures and other aspects of the spray drying process. Excessive degradation of the pharmaceutical agent can lead to drug formulations lacking the requisite purity and a risk of loss in bioactivity of the pharmaceutical agent.

Another technical hurdle which must be overcome in the application of spray drying technology to the field of pulmonary drug administration is the particular sizing requirements necessary to administer the resultant particles to the deep lung. For pulmonary applications, the aerodynamic size of the particles dispersed in an aerosol directly impacts the deposition pattern in the lung. In order for a particle to be considered respirable (i.e. capable of administration to the alveoli of the deep lung), the mass median aerodynamic diameter (MMAD) should be maintained below 10 *m, preferably in the range of 0.4-5 μm, and the amount of the composition comprising particles outside of this target size range should be minimized.

The major factors influencing this final particle size are the initial liquid drop size, the initial solids concentration, and the drying rate. The processing economics are directly impacted by the solids concentration in the feed stock; the lower the concentration, the more cost associated with driving off the solvent per unit mass of recovered product. Therefore, it is advantageous to create small liquid droplets with the highest concentration feasible for a particular process to minimize capital equipment and operating costs.

The ability to control the droplet size distribution has been theorized as being beneficial based upon the need to concentrate the particle mass in the target size range, and minimize or eliminate the fraction of the product that is outside of the respirable range or ‘fines’, i.e. particles of typically less than 0.4 μm diameter. The ability to create a narrow droplet size distribution in the appropriate size range provides control of the initial evaporation rate. In addition, a reduction in the percentage of ‘fines’ in the bulk particle size distribution would improve the overall process efficiency and allow for the more efficient use of cyclone separators to collect the dried particles and increase process yield.

Further, the ability to produce controlled multimodal powders could significantly impact the dispersibility of the final particles. For example, powder consisting of a narrow size distribution of particles in the respirable range combined with a small fraction (i.e. less than 2% of the total mass) of ‘fines’ could significantly reduce the effects of interparticle cohesion between the larger particles and facilitate bulk powder flowability, as well as dispersibility of the powder in a dry powder inhaler (DPI). Thus, the ability to produce multimodal particles containing different populations of discrete particle sizes in a one-step process (i.e. without the need for size classification) may be advantageous for delivery of particles to the deep lung by aerosolization.

Additionally, the ability to engineer the primary particle size distribution in the range of interest for pulmonary use could have an impact on the resulting bio-availability of the product by targeting specific deposition sites. This effect could also be enhanced by in-process blending of different medications with specific particle sizes assigned to each.

Spray drying dry powder pharmaceuticals is known, but has usually been limited to spray drying of small molecules and other stable drugs which are less sensitive to thermal degradation, and most commonly hydrophilic drugs in aqueous solutions. For example, U.S. Pat. Nos. 5,260,306, 4,590,206, GB 2,105,189, and EP 072 046 describe a method for spray drying nedocromil sodium to form small particles preferably in the range from 2-15 μm for pulmonary delivery. U.S. Pat. No. 5,376,386 describes the preparation of particulate polysaccharide carriers for pulmonary drug delivery, where the carriers comprise particles sized from 5-100 μm and having a rugosity of less than 1.75. WO 96/09814 discloses spray-dried smooth and spherical microparticles which either carry a therapeutic or diagnostic agent. U.S. Pat. No. 6,022,525 discloses microcapsules prepared by spray-drying and which are useful for ultrasonic imaging. Additionally, aerodynamically light particles for pulmonary delivery and particles incorporating surfactants for pulmonary drug delivery and their preparation are disclosed in U.S. Pat. Nos. 5,855,913 and 5,874,064.

The spray drying of hydrophobic drugs and excipients is disclosed in U.S. Pat. Nos. 5,976,574, 5,985,248, 6,001,336, and 6,077,543, all of which are hereby incorporated in their entirety by reference. Additional spray drying processes are disclosed in EP 1004349, WO 96/32149, WO 99/16419, and U.S. Pat. Nos. 6,000,241, and 6,051,256, and in The Spray Drying Handbook, K. Masters, which are hereby incorporated in their entirety by reference. The optimization of the physical and chemical characteristics of spray dried materials can involve the adjustment of processing parameters such as inlet drying temperature, outlet drying temperature, feed spray rate, atomizing pressure, air flow volume, or atomizer type. Additionally, a number of variables including the droplet size and distribution, the inlet temperature of the gas stream, the outlet temperature of the gas stream, the inlet temperature of the liquid droplets, and the manner in which the atomized spray and hot drying gas are mixed, may be controlled in order to control the drying rate. Control of parameters such as the drying rate, solids concentration, and flow rates can also influence particle morphology.

Various atomizers have been used in the spray drying of pharmaceutical powders. These include gas assisted two fluid nozzles, rotary atomizers and ultrasonic atomizers comprising an oscillating horn to create surface instabilities resulting in droplet formation. Examples of each of these various atomizers are disclosed in the patents cited above. Droplet size and droplet size distribution are determined by the selection of the atomizer.

Sonic air-assisted two fluid atomization nozzles (two fluid nozzles) involve impacting liquid bulk with high velocity gas, utilizing the kinetic energy of the gas stream to create the liquid surface area. Sprays of low viscosity feed are characterized by low mean droplet sizes. Formation of sprays having a mass median diameter of 15-20 microns are well established for such two fluid nozzles. With more viscous feeds, larger mean droplet sizes are produced with a wider particle size distribution. Among the variables affecting mean droplet size for two fluid nozzles, the mass ratio (Mair:Mliq) and design details of the given atomizer (e.g. prefiliming or regular) are perhaps the most important variables.

A significant amount of energy is required to generate the high velocity gas stream necessary to atomize a feed stock with a two fluid nozzle. Two fluid nozzles utilize a high pressure ratio to generate the high velocity gas stream. As a result, a cooler gas (relative to the hot drying gas) exits the two fluid nozzle. This shroud of cool gas surrounds the atomized spray exiting the two fluid nozzle. This results in a disparity in drying conditions experienced by the droplets, as droplets located near the center of this cloud are exposed to a different drying environment compared to droplets located at the interface of the atomizer spray and drying gas. This disparity in near-nozzle drying conditions affords less control over process parameters to control the final powder characteristics than could be possible if the effects of atomization gas could be minimized or mitigated.

In rotary atomization, the feed liquid is centrifugally accelerated to high velocity before being discharged into an air-gas atmosphere. The liquid is distributed centrally on a rotating wheel/disc/cup and extends over the rotating surface as a thin film. Operating variables that influence droplet size produced from atomizer wheels are speed of rotation, wheel diameter, wheel design (number and geometry of vanes or bushings), feed rate, viscosity of feed and air, density of feed and air, and surface tension of feed. Two fluid nozzles are capable of producing smaller droplets compared to rotary atomizers. Typical droplet size distributions for two fluid nozzles are depicted in FIGS. 5-7. It is perhaps for their ability to produce smaller droplets that two-fluid nozzles are currently more commonly used in spray drying applications for producing particles for pulmonary administration.

More recently, interest has focused on electrically assisted ultrasonic atomizers. Such interest has been prompted by the need to develop a technique to atomize products that are non Newtonian, highly viscous, and have long chain molecular structures, and that form only strings or filaments from rotary atomizers and liquids that require very high pressure for effective atomization from pressure nozzles. One recent study compared atomizer performance in the production of respirable spray-dried particles using a two fluid nozzle atomizer and an ultrasonic atomizer. Dunbar et al. “Evaluation of Atomizer Performance in Production of Respirable Spray-Dried Particles”, Pharmaceutical Development and Technology, pp. 433-441 (1998). The study concluded that the two fluid atomizer produced smaller droplets (Sauter mean diameter=4.5 -4.8 *m) relative to the ultrasonic nebulizer (Sauter mean diameter=22-48*m). Additionally, the results showed that the ultrasonic nebulizer performed poorly as feed stock flow rates increased beyond 3 ml/mn.

The use of ultrasonically assisted pressure atomization to obtain narrow droplet size distributions is known in the field of ink-jet printing. While this area generally deals with much larger drops (approximately 60 microns) at relatively low mass flowrates, precise control of the drop size is common to ink-jet printing since it effects image quality. However, the use of ultrasonic atomizers in the ink-jet field are not concerned with problems associated with the manufacture of pharmaceuticals including proteins and peptides and the requirements to produce respirable particles as discussed above. The above describes some of the problems currently encountered in the development of spray drying processes for pharmaceutical application, particularly with respect to spray drying powders for pulmonary administration. Moreover, it can be difficult to achieve a desired low moisture content required for physical and chemical stability in the final particulate product, particularly in an economic manner. Finally and perhaps most importantly, it has been difficult to produce the small particles necessary for pulmonary delivery in an efficient manner on a large scale suitable for commercial applications.

In view of the above, there remains a need to provide improved process control over the ultimate particle size and particle size distribution, particularly with respect to the production of particles suitable for pulmonary drug administration. The present invention overcomes the above problems found in the prior art and provides an improved process for producing particles for pulmonary delivery.

### DESCRIPTION OF TERMS

“Active agent” as described herein includes an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. The active agent that can be delivered includes antibiotics, antiviral agents, anepileptics, analgesics, anti-inflammatory agents and bronchodilators, and viruses and may be inorganic and organic compounds, including, without limitation, drugs which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, polysaccharides, steroids, hypnotics and sedatives, psychic energizers, tranquilizers, anticonvulsants, muscle relaxants, antiparkinson agents, analgesics, anti-inflammatories, muscle contractants, antimicrobials, antimalarials, hormonal agents including contraceptives, sympathomimetics, polypeptides, and proteins capable of eliciting physiological effects, diuretics, lipid regulating agents, antiandrogenic agents, antiparasitics, neoplastics, antineoplastics, hypoglycemics, nutritional agents and supplements, growth supplements, fats, antienteritis agents, electrolytes, vaccines and diagnostic agents. Examples of active agents useful in this invention include but are not limited to insulin, calcitonin, erythropoietin (EPO), Factor VIII, Factor IX, ceredase, cerezyme, cyclosporine, granulocyte colony stimulating factor (GCSF), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-2, luteinizing hormone releasing hormone (LHRH, somatostatin, somatostatin analogs including octreotide, vasopressin analog, follicle stimulating hormone (FSH), insulin-like growth factor, insulintropin, interleukin-1 receptor antagonist, interleukin-3, interleukin-4, interleukin-6, macrophage colony stimulating factor (M-CSF), nerve growth factor, parathyroid hormone (PTH), thymosin alpha 1, IIb/IIIa inhibitor, alpha-1 antitrypsin, respiratory syncytial virus antibody, cystic fibrosis transmembrane regulator (CFTR) gene, deoxyribonuclease (Dnase), bactericidal/permeability increasing protein (BPI), anti-CMV antibody, interleukin-1 receptor, 13-cis retinoic acid, pentamidine isethionate, albuterol sulfate, metaproterenol sulfate, beclomethasone dipropionate, triamcinolone acetamide, budesonide acetonide, ipratropium bromide, flunisolide, fluticasone, cromolyn sodium, ergotamine tartrate and the analogues, agonists and antagonists of the above. Active agents may further comprise nucleic acids, present as bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells, particularly cells of the alveolar regions of the lungs. The active agents may be in various forms, such as soluble and insoluble charged or uncharged molecules, components of molecular complexes or pharmacologically acceptable salts. The active agents may be naturally occurring molecules or they may be recombinantly produced, or they may be analogs of the naturally occurring or recombinantly produced active agents with one or more amino acids added or deleted. Further, the active agent may comprise live attenuated or killed viruses suitable for use as vaccines.

“Mass median aerodynamic diameter” or “MMAD” is a measure of the aerodynamic size of a dispersed aerosol particle. The aerodynamic diameter is used to describe an aerosolized particle in terms of its settling behavior, and is the diameter of a unit density sphere having the same settling velocity, generally in air, as the particle in question. The aerodynamic diameter encompasses particle shape, density and physical size of a particle. As used herein, MMAD refers to the midpoint or median of the aerodynamic particle size distribution of an aerosolized particle determined by cascade impaction.

“Mass median diameter” or “MMD” is a measure of mean particle size. Any number of commonly employed techniques can be used for measuring mean particle size.

As used herein, “monodisperse” refers to a collection of particles (bulk or aerosol dispersion) comprising particles of a substantially uniform MMD.

As used herein, “multimodal” refers to a collection of particles (bulk or aerosol dispersion) of at least two distinct populations wherein each subpopulation of particles is characterized by having a substantially uniform MMD.

As used herein, “particle” refers to liquid droplets as well as to dry particulates.

As used herein, “physiologically effective amount” refers to that amount delivered to a subject to give the desired palliative or curative effect. This amount is specific for each drug and its ultimate approved dosage level.

As used herein, the term “pulmonary administration” refers to the delivery of an agent to the pulmonary passages of a subject for local or systemic delivery such as by inhalation, nasal administration, nebulization, ventilation, and the like.

As used herein, “therapeutically effective amount” refers to the amount present in the composition that is needed to provide the desired level of drug in the subject to be treated to give the anticipated physiological response. This amount is determined for each drug on a case-by-case basis.

### SUMMARY OF THE INVENTION

The present invention is directed to a process and apparatus to produce particles of a given particle size and particle size distribution, as well as the particles made thereby. The accurate and reproducible control of the droplet size and droplet size distribution from an atomizer used to produce a droplet spray according to the instant invention enables the production of particles with tight particle size distribution. The particles of the present invention are particularly suited for pulmonary drug administration, although the invention can be practiced in other fields such as known in the chemical and food industries.

Although the detailed description describes a preferred embodiment directed to spray drying, it is to be understood that the technology of the present invention can be used in other ways to produce dry particles or aerosolize liquid particles. For example, the apparatus and methods of the present invention can be used in combination with devices of the type disclosed in U.S. Pat. Nos. 5,938,117 and 6,014,970, hereby incorporated in their entirety by reference, to produce the aerosolized spray of liquid feed stocks as disclosed therein. The apparatus and methods of the present invention can also be used in a variety of methods known in the art to produce particles from a liquid feed stock. For example, the present invention can be used in super critical fluid processing techniques as disclosed in U.S. Pat. Nos. 5,851,453 and 6,063,138, hereby incorporated in their entirety by reference, and with spray congealing methods as disclosed in U.S. Pat. No. 5,727,333, hereby incorporated in its entirety by reference.

According to a preferred embodiment, the particles are produced by spray drying the liquid feed stock in order to produce a desired particle size and particle size distribution of the spray dried particles. In particular, a preferred embodiment of the present invention provides methods for spray drying and particles produced thereby wherein the method provides spray drying process control which produces dry particles having a narrow particle size distribution suitable for pulmonary administration. According to this embodiment, spray dried particles can be produced having a desired median diameter and particle size distribution resulting solely from the spray drying process. Further separation processing, such as filtration or centrifugation and the like, is not necessary to provide the desired particle size distribution. Control of particle size and particle size distribution of the present invention can be used in combination with control over other process parameters, such as drying rate, to provide even more control over particle morphology.

The methods of the present invention are useful for producing particles of pharmaceutical agents such as proteins, polypeptides, oligopeptides, nucleic acids, and the like. The method is particularly useful for the production of particles of a size suitable for pulmonary administration.

Compositions according to the present invention comprise dispersible particles intended for pulmonary administration, i.e., inhalation by a patient into the alveolar regions of the patient's lungs. The compositions comprise particles having MMAD below 10 μm with at least 70% of the mass of the particles having a diameter within a 4 μm range.

Accordingly, it is an aspect of this invention to provide a method for controlling particle size and particle size distribution of spray dried particles, particularly for spray dried particles intended for pulmonary administration.

It is another aspect of this invention to provide a method for spray drying particles of a size suitable for pulmonary administration wherein the atomizer spray droplet size and distribution is controlled in order to achieve a desired spray dried particle size and particle size distribution.

It is another aspect of this invention to provide a substantially monodisperse droplet size distribution from an atomizer.

It is yet another aspect of this invention to provide an atomizer droplet size distribution wherein at least 80% of the droplet mass is in droplets having a diameter within ±25% of the median droplet diameter, wherein the median droplet diameter is less than 40 μm.

It is another aspect of this invention to provide a spray drying process which is economical for commercial scale production at flow rates in excess of 3 ml/mn.

These and other aspects of the present invention will be readily apparent to one of ordinary skill in the art in view of the following description and examples.

### BRIEF DESCRIPTION OF THE DRAWINGS

FIG. 1 is a block diagram illustrating the primary unit operations of the methods of the present invention.

FIG. 2 is a cross-section of an atomizer according to one embodiment of the present invention.

FIGS. 3a and 3b are top views of the atomizer nozzle plate according to the present invention.

FIG. 4 depicts an array of atomizers according to the invention.

FIGS. 5-7 depict plots of the droplet size distribution from two twin-fluid nozzles.

FIG. 8 depicts a plot of the droplet size distribution from an ultrasonic atomizer according to the present invention.

FIG. 9 is a SEM image of spray dried particles using a twin-fluid atomizer.

FIG. 10 is a SEM image of spray dried particles produced according to this invention at a fast drying rate.

FIG. 11 is a SEM image of spray dried particles produced according to this invention at a slow drying rate.

FIG. 12 depicts a plot comparing particle size distribution of particles produced using twin fluid atomizer and an ultrasonically assisted atomizer according to the invention.

### DETAILED DESCRIPTION OF THE INVENTION

According to a preferred embodiment, the present invention relates to methods for spray drying compositions containing a pharmaceutical agent to produce dry powders intended primarily for pulmonary administration to patients for a variety of therapeutic and clinical purposes. A first aspect of the invention relates to control of particle characteristics which enable use of the particles for the intended purposes. A second aspect of the invention is directed to the capacity of the demonstrated process to produce particles with the desired characteristics at a scale that can support market requirements for a given drug.

According to a preferred embodiment of the invention, accurate and reproducible control of the spray dryer atomizer droplet size distribution is provided which results in the production of spray dried particles having narrow particle size distributions. The present invention is directed to particular atomizer spray characteristics which result in the production of particles suitable for pulmonary administration. The spray dryer atomizer is selected so as to produce a spray of droplets having a median diameter of less than 40 microns, preferably less than 20 microns and most preferably less than 11 microns.

For the production of particles for pulmonary administration, the atomizer is selected to produce a droplet size distribution effective to yield particles wherein at least 70% of the mass of the dry solid particles, preferably at least 80%, more preferably at least 90%, and most preferably at least 95%, have a particle size distribution of within 4 microns, preferably within 3 microns, and most preferably within 1.5 microns, without requiring separation processing for the droplets or the dry particles. According to one embodiment of the invention, the atomizer droplet size distribution is substantially monodisperse in order to produce substantially monodisperse dry particles. According to this embodiment, the atomizer produces a droplet size distribution wherein at least 80% of the droplets, preferably at least 90%, and most preferably at least 95% of the droplets have a diameter within ±25% of the median droplet diameter, preferably within ±15%, and most preferably within ±8% of the median droplet diameter.

According to another embodiment, the atomizer droplet size distribution is controlled to produce a multimodal collection of particles with a predetermined particle size and particle size distribution. According to this embodiment, multiple populations of particles having distinct particle size distributions are provided. These multiple populations may be formed from the same or different feed stock formulations. For example, a multimodal distribution according to this embodiment may comprise active agent containing particles having a first MMAD in the respirable range and a second population of particles without any active agent which may have a MMAD within or outside of the respirable range. Alternatively, the second population of particles may comprise the same or a different active agent than the first population. Additionally, multimodal distributing according to this invention are not limited to only 2 distinct populations but may include as many distinct populations as desired as will be understood from the teachings herein. Thus, according to the present invention, multimodal particles can be produced in a single step.

Additionally, the atomizer is selected so as to encompass liquid flowrates of preferably greater than 5 ml/min and up to several 1/min suitable for commercial scale production. The liquid medium may be a suitable solution, suspension, emulsion, or other dispersion of the pharmaceutical agent in a suitable liquid carrier. Suitable liquid carriers include water and other organic liquids such as ethanol and the like.

According to the invention, the atomizer produces droplets with a desired median diameter of less than 40 μm, preferably less than 20 μm, and more preferably less than 11 μm. Additionally, according to the invention, the atomizer is selected such that it produces fine droplets having a narrower particle size distribution then previously available without requiring any sizing classification or separation. It is the ability to set a droplet size and control the droplet size distribution without requiring any size classification or separation provided by this invention which provides a much improved process control over final particle size and particle size distribution in such applications which heretofore has not been possible with current atomizers such as twin fluid nozzles and rotary atomizers.

According to the invention, any atomizer capable of providing the desired droplet size and provide droplet size distribution wherein at least 80% of the droplets, preferably at least 90%, and most preferably at least 95% of the droplets, is in droplets having a diameter within ±25% of the median droplet diameter, preferably within ±15%, and most preferably within ±8% of the median droplet diameter is suitable for use with the present invention. These distributions are preferably measured on a mass basis. A preferred atomizer for use in the present invention is the droplet generator described in U.S. Pat. No. 5,248,087, herein incorporated in its entirety by reference.

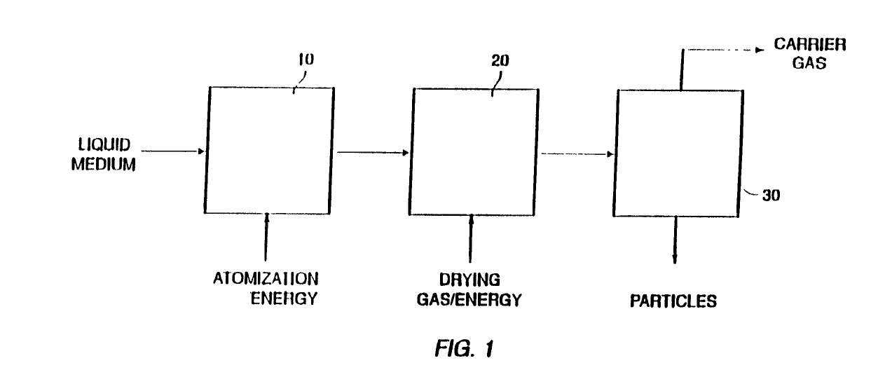

Referring now to FIG. 1, a preferred embodiment of the present invention directed to a spray drying process for preparing dispersible dry powders of a pharmaceutical agent will be described. The spray drying process comprises an atomization operation 10 which produces droplets of a liquid medium having a droplet size distribution as discussed above which are dried in a drying operation 20. Drying of the liquid droplets results in formation of the discrete particles which form the dry powder compositions which are then collected in a separation operation 30. Each of these unit operations will be described in greater detail below.

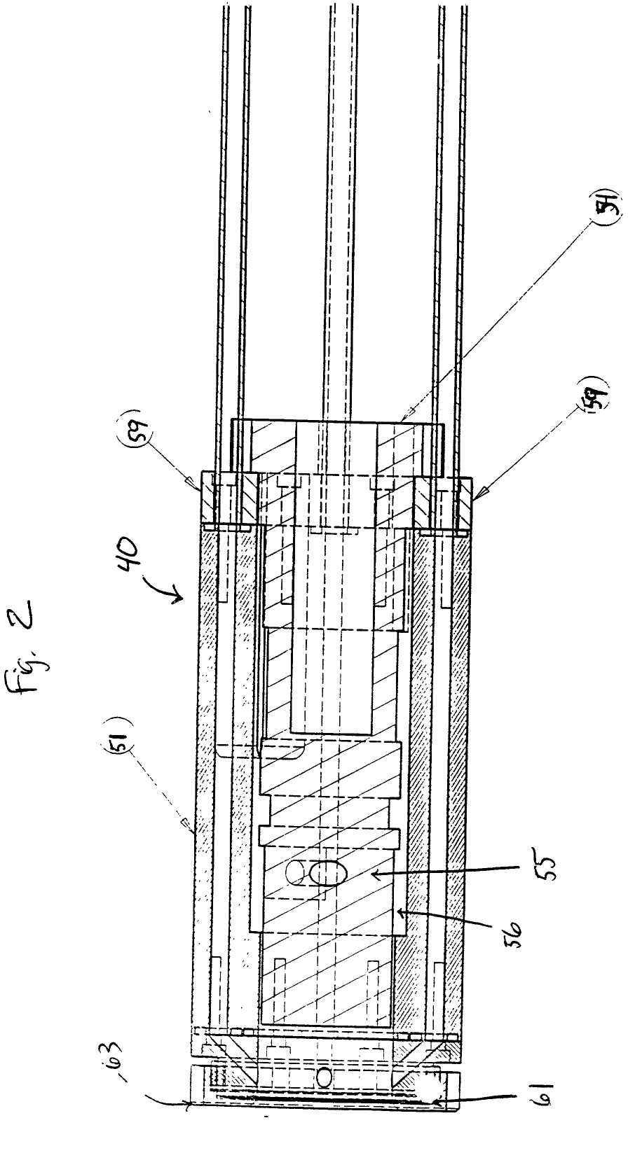

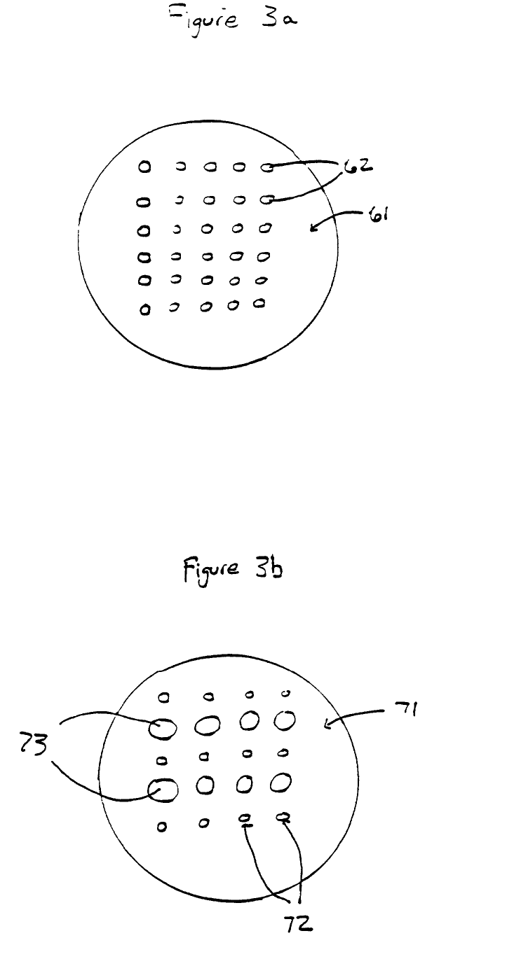

An atomizer 40 for producing a substantially monodisperse droplet distribution for a spray drying apparatus in accordance with a preferred embodiment of the present invention is shown in FIG. 2. The atomizer 40 includes a housing 51 having a substantially cylindrical main body portion. Acoustic transducer 54 is connected to the main body portion of the housing 51. The transducer 54 includes a piston 55 within an inner cavity 56 of the housing 51. A feed stock communicates with the acoustic transducer 54 through a liquid feed assembly 59. A drive means (not shown) is connected to the transducer 54 for driving the transducer 54 and causing the piston 55 to impart acoustic energy to the fluid thereby creating high amplitude velocity perturbations on the outgoing fluid stream which are sufficient to atomize the fluid into a stream of droplets. The fluid exits from the atomizer via orifices or nozzles 62 formed within a plate 61 depicted in FIGS. 3a and 3b. Plate 61 is held in position by retainer 63.

As seen in FIG. 3a plate 61 comprises an array of orifices 62. It is to be understood that the array can be in a different geometrical configuration in order to produce different spray characteristics. For a substantially monodisperse spray, a constant orifice diameter is selected. Suitable orifice diameters to produce particles suitable for pulmonary administration are less than 30 μm, preferably less than 20 μm, and most preferably less than 10 μm.

FIG. 3b depicts plate 71 suitable for practicing another embodiment of the invention directed to multimodal atomizer sprays for the production of spray dried particles having a multimodal distribution. As seen in FIG. 3b, plate 71 comprises an array of orifices 72, 73 including orifices of a first diameter 72 and orifices of at least a second diameter 73, such that droplets are produced having a distribution of droplets of at least two different diameters. According to this embodiment, the first diameter is less than 30 μm preferably less than 20 μm, and most preferably less than 10 μm and the second diameter is within the range of ±50% of the first diameter, preferably within ±30% of the first diameter, and most preferably within ±20% of the first diameter. It is to be understood that any number of different orifice diameters can be selected so as to provide a spray with the desired number of distinct droplet diameters. It is further understood that the orifice geometries are not to be limited to circular configurations, but may be any desired shape such as diamond, cross-shaped, T-shaped and the like. The orifices can be made by processes known in the art such as laser drilling and photo-etching.

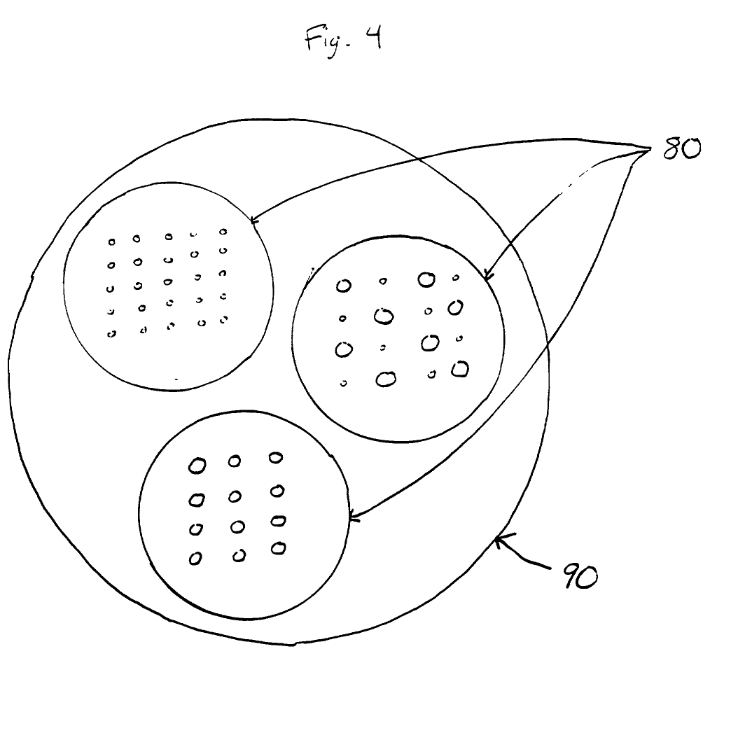

According to another embodiment depicted in FIG. 4, a multimodal droplet distribution is produced by providing an array of atomizers 80 supported by a mounting ring 90. The array of atomizers 80 is provided such that at least one of the atomizers 80 produces droplets having a droplet size and droplet distribution different from the at least one other atomizer in the array. For example, each of the nozzle plates of the atomizers 80 may comprise different orifice diameters as depicted in FIG. 4. Any combination of diameters, geometric configurations of orifice array, as well as number of atomizers are contemplated as within the scope of this invention. Multimodal distributions can be produced by means other than altering the geometries of nozzle plate and array of nozzles. For example, multimodal distributions can be made by altering the frequency of the atomizer as well as adjusting the solids concentration in the feed stock, for example.

The drying operation is controlled to provide dried particles having particular characteristics, such as a rugosity above 2 as described in WO 97/41833 cited above. The drying rate may be controlled by a number of variables, including the droplet size distribution, the inlet temperature of the gas stream, the outlet temperature of the gas stream, the inlet temperature of the liquid droplets, and the manner in which the atomized spray and drying gas are mixed. Preferably, the drying gas stream will have an inlet temperature of at least 90° C., preferably at least 120° C., and more preferably at least 135° C., and still more preferably at least 145° C. and often 175-200° C. depending upon the particular active agent being treated.

In order to control the final moisture content of the particles produced in the drying operation, it is desirable to also control the gas outlet temperature and or relative humidity. The gas outlet temperature will be a function of the inlet temperature, the heat load imposed by the product drying step (which depends on the inlet temperature of the liquid medium, the quantity of water to be evaporated, and the like), and other factors. Preferably the gas outlet temperature will be maintained at least 50° C. or above, preferably at least 70° C., usually in the range from 60-80° C.

In yet another aspect of the method of the present invention, the drying conditions will be selected to control the particle morphology. According to this aspect of the invention, higher drying rates are used to produce particles having highly irregular, dimpled surfaces. Such particles preferably have a rugosity greater than 2. Higher drying rates according to this aspect of the invention are characterized by an inlet drying temperature of at least 100° C., preferably of at least 125° C. and an outlet drying temperature of less than 100° C., preferably less than 90° C. Particles characterized by a more spherical, uniform surface may be produced by using slower drying rates. The combination of control over droplet size and control over drying rate according to this invention provides control over particle morphology.

The separation operation 30 will be selected in order to achieve very high efficiency collection of the particles produced by the drying operation 20, as described in WO 97/41833 cited above.

Preferred compositions according to this invention comprise dispersible powders intended for pulmonary delivery, i.e., inhalation by a patient into the alveolar regions of the patient's lungs. It is also contemplated that the spray drying process of this invention can be utilized in spray drying other products where narrow particle size distributions, i.e. within a range of 4 μm or less, are desired. According to the preferred embodiment directed to spray drying particles for pulmonary administration, the compositions preferably comprise particles having a MMAD below 10 μm. According to this embodiment, at least 70% of the mass of the particles, preferably at least 80%, and more preferably at least 90%, will comprise particles having a particle size within a 4 μm range or less, preferably with a 3 μm range and most preferably within a 1.5 μm range. According to a particularly preferred embodiment, at least 95% of the mass of the composition will comprise particles having a particle size within the above ranges. The compositions will often be packed as unit doses where a therapeutically effective amount of the composition is present in a unit dose receptacle, such as a blister pack, gelatin capsule, or the like. The spray dried powders for pulmonary administration of the present invention can be incorporated into such unit dose forms without further size classification, and no need for secondary steps for blending or homogenization of the distribution.

A pharmaceutically acceptable excipient may optionally be incorporated into the particles (or as a bulk carrier for the particles) to provide the stability, dispersibility, consistency, and/or bulking characteristics to enhance uniform pulmonary delivery of the composition to a subject in need thereof. The amount of excipient may be up to about 99.95% w, depending on the activity of the drug being employed. Preferably about 5%w to about 95% w will be used. Such excipients may serve simply as bulking agents when it is desired to reduce the active agent concentration in the powder which is being delivered to a patient. Such excipients may also serve to improve the dispersibility of the powder within a powder dispersion device in order to provide more efficient and reproducible delivery of the active agent and to improve the handling characteristics of the active agent (e.g., flowability and consistency) to facilitate manufacturing and powder filling. In particular, the excipient materials can often function to improve the physical and chemical stability of the active agent, to minimize the residual moisture content and hinder moisture uptake, and to enhance particle size, degree of aggregation, surface properties (i.e., surface energy, rugosity), ease of inhalation, and targeting of the resultant particles to the deep lung. Alternatively, the active agent may be formulated in an essentially neat form, wherein the composition contains active agent particles within the requisite size range and substantially free from other biologically active components, pharmaceutical excipients, and the like. Pharmaceutical excipients and additives useful in the present composition include but are not limited to proteins, peptides, amino acids, lipids, polymers, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which may be present singly or in combination. Exemplary protein excipients include serum albumin such as human serum albumin (HSA), recombinant human albumin (rHA), gelatin, casein, and the like. Representative amino acid/polypeptide components, which may also function in a buffering capacity, include alanine, glycine, arginine, betaine, histidine, glutamic acid, aspartic acid, cysteine, lysine, leucine, proline, isoleucine, valine, methionine, phenylalanine, aspartame, and the like. Polyamino acids of the representative amino acids such as di-leucine and tri-leucine are also suitable for use with the present invention. One preferred amino acid is leucine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. The dry powder compositions may also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additionally, the dry powders of the invention may include polymeric excipients/additives such as polyvinylpyrrolidones, hydroxypropyl methylcellulose, methylcellulose, ethylcellulose, Ficolls (a polymeric sugar), dextran, dextrates (e.g., cyclodextrins, such as 2-hydroxypropyl-b-cyclodextrin, hydroxyethyl starch), polyethylene glycols, pectin, flavoring agents, salts (e.g. sodium chloride), antimicrobial agents, sweeteners, antioxidants, antistatic agents, surfactants (e.g., polysorbates such as “TWEEN 20” and “TWEEN 80”, lecithin, oleic acid, benzalkonium chloride, and sorbitan esters), lipids (e.g., phospholipids, fatty acids ), steroids (e.g., cholesterol), and chelating agents (e.g., EDTA). Other pharmaceutical excipients and/or additives suitable for use in the compositions according to the invention are listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are herein incorporated by reference.

According to the present invention, a dispersing agent for improving the intrinsic dispersibility properties of the powders may also be added. Suitable agents are disclosed in PCT applications WO 95/31479, WO 96/32096, and WO 96/32149, hereby incorporated in their entirety by reference. As described therein, suitable agents include water soluble polypeptides and hydrophobic amino acids such as tryptophan, leucine, phenylalanine, and glycine. Leucine is particularly preferred for use according to this invention.

The following examples are offered by way of illustration, not by way of limitation.

### EXAMPLE 1

Liquid droplet size distributions from two different twin-fluid atomizers were studied. Liquid droplet data was collected using a phase Doppler particle analyzer. The atomizers were used with Niro spray dryers. The atomizer operating conditions are listed in Table 1 and the liquid sprayed was water only.

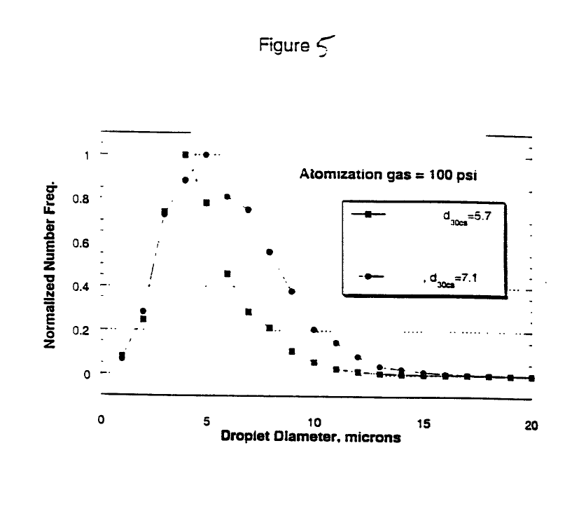

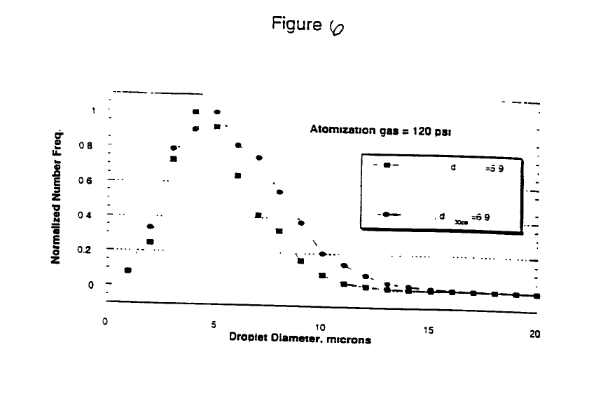

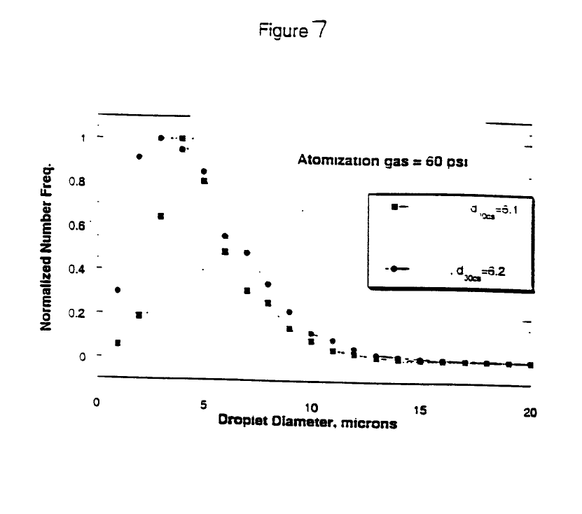

FIGS. 5-7 show the cross-sectional size distributions for both atomizer designs at 60, 100 and 120 psig. atomization gas pressure, respectively.

TABLE 1
Atomizer Operating Conditions

| | Liquid flow, | Gas pressure, | Measurement |
|---|---|---|---|
| Atomizer | ml/min | PSIG. | distance, in. |
| 1 | 50 | 60 | 7 |
| 1 | 50 | 100 | 7 |
| 1 | 50 | 120 | 7 |
| 1 | 90 | 60 | 7 |
| 1 | 90 | 100 | 7 |
| 1 | 90 | 120 | 7 |
| 2 | 50 | 60 | 7 |
| 2 | 50 | 100 | 7 |
| 2 | 50 | 120 | 7 |
| 2 | 90 | 60 | 7 |
| 2 | 90 | 100 | 7 |
| 2 | 90 | 120 | 7 |

### EXAMPLE 2

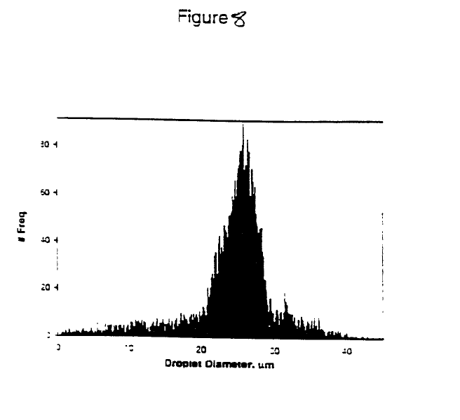

Water pressurized to 20 psig. was delivered to the atomizer feed circuit of the atomizer depicted in FIG. 2. The electronic transducer was energized using a standard function generator providing a sign wave at 195 kHz at 20 volts peak to peak amplitude. The resulting liquid droplet diameters were measured in the spray using a commercially available Phase Doppler Particle Analyzer. The droplet particle size distribution from the ultrasonic pressure assisted atomizer is depicted in FIG. 8.

### EXAMPLE 3

Feed stock solutions containing alpha-1 antitrypsin were prepared by mixing alpha-1-antitrypsin (Aventis Behring) with water to provide a solids content of about 1.5-3%. The alpha-1-antitrypsin solutions were diluted with water to a solids content of 0.1% and spray dried on a Niro spray dryer using the ultrasonic atomizer depicted in FIG. 2. The 1.5-3% solids alpha-1-antitripsin solutions were also spray dried under similar conditions on Niro spray driers using the twin fluid nozzles of Example 1. The spray dryer conditions are set forth in Table 2.

TABLE 2
Spray Dryer Operating Conditions

| Total | | | | | RH % @ T |
|---|---|---|---|---|---|
| Solids % | Liq ml/min | Air, SCFM | T in C | T out C | out |
| 0.1 | 20 | 28 | 155 | 80 | 8.9 |
| 0.1 | 20 | 28 | 135 | 65 | 16.9 |

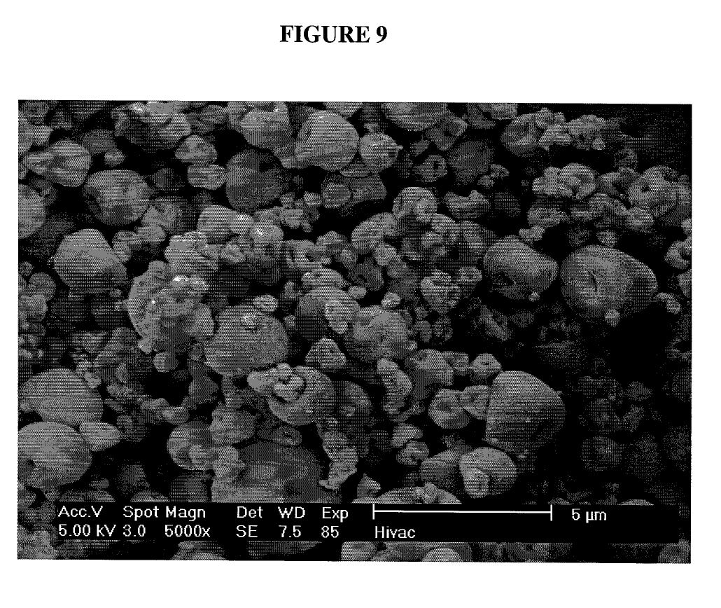

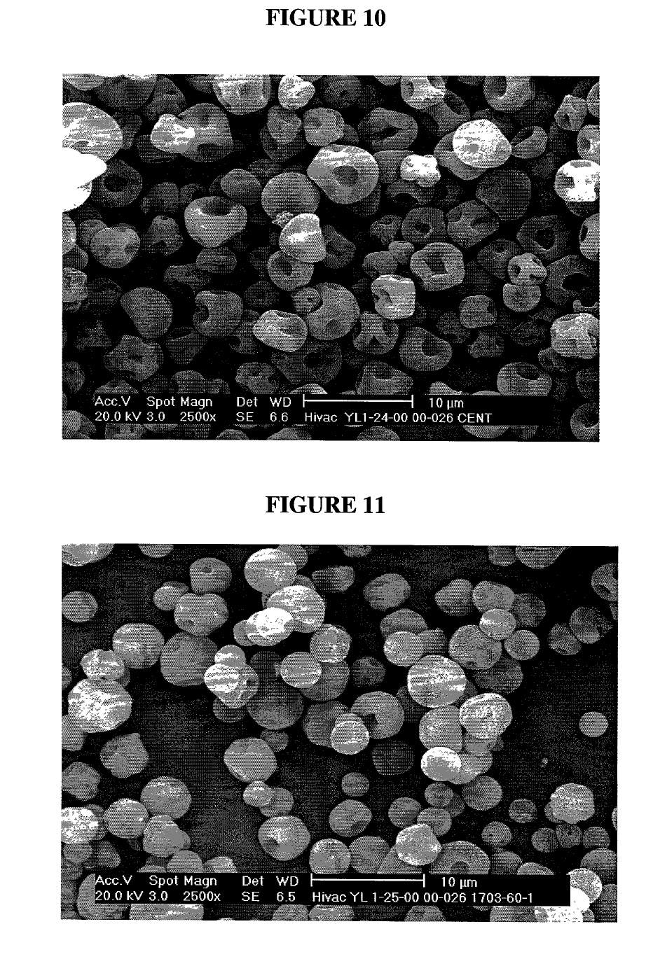

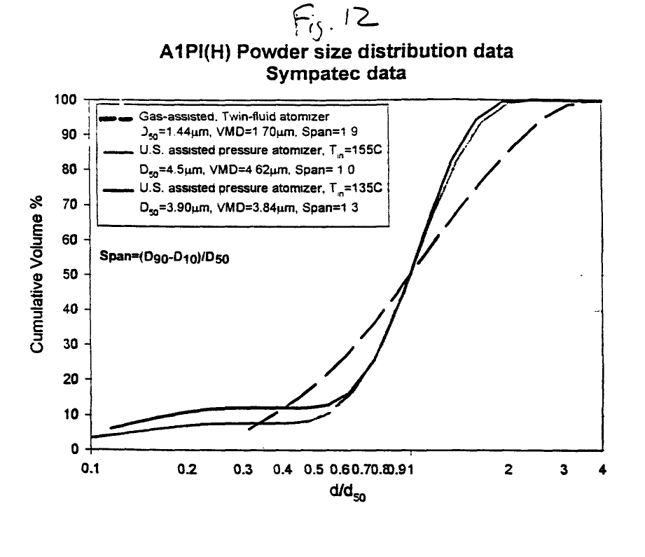

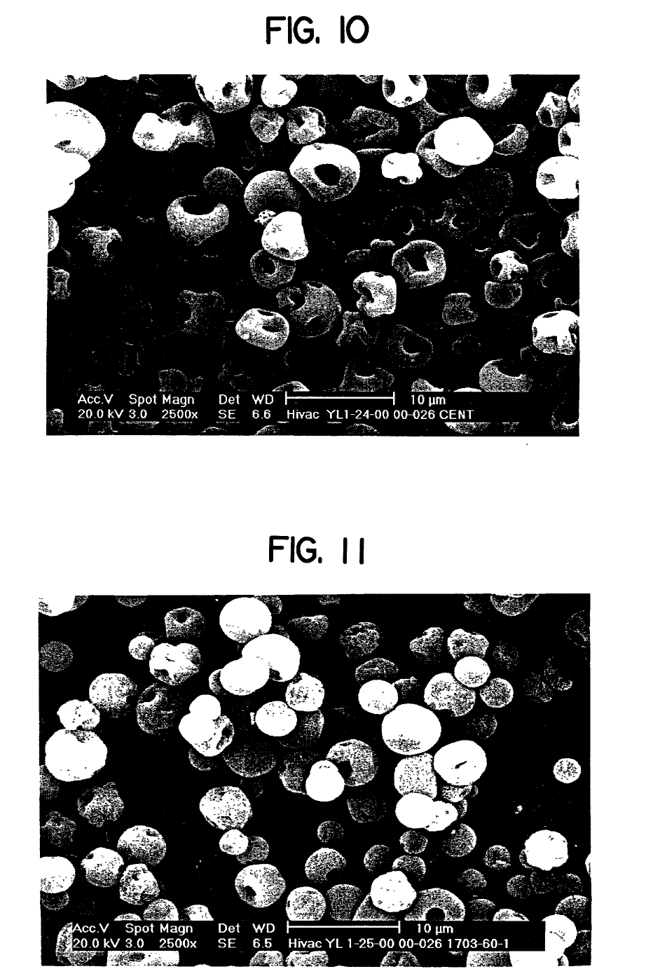

FIG. 9 depicts SEMs obtained for powders produced by spray drying with the twin fluid nozzle. FIG. 10 depicts SEM images obtained for powders produced at the faster drying conditions (RH 8.9a) and FIG. 11 depicts SEM images obtained for powders produced at the slower drying conditions (RH 16.9). The particles produced using the ultrasonic atomzer had a narrower size distribution as seen in FIG. 12 and more uniform morphology as seen in the SEMs compared to those produced using the twin fluid nozzle.

We claim:

1. A method for making particles from a liquid feed stock containing a pharmaceutical agent to produce particles suitable for pulmonary administration having a narrow particle size distribution comprising:
providing a feed stock comprising a pharmaceutically active agent and a solvent;
forcing said feed stock into a manifold defined between a vibratable element and a plate and forcing the feed stock through the plate, said plate comprising holes of at least one predetermined diameter, in order to produce droplets comprising a droplet size distribution wherein at least 80% of the droplets have a diameter within ±25% of the median droplet diameter;
removing solvent from said droplets to produce particles suitable for pulmonary administration.

2. A method according to claim 1 further comprising vibrating said vibratable element in order to force said feed stock through the plate and produce droplets.

3. A method according to claim 2 wherein a piezoelectric element is coupled to said vibratable element.

4. A method according to claim 1 wherein said holes comprise a predetermined diameter of less than 30 microns.

5. A method according to claim 4 wherein said holes comprise a predetermined diameter of less than 10 microns.

6. A method according to claim 1 wherein said plate comprises holes having a first diameter of less than 30 microns and a second series of holes having a second diameter of ±50% of said first diameter.

7. A method according to claim 6 wherein said second diameter is within ±20% of said first diameter.

8. A method according to claim 7 wherein said first diameter is less than 10 microns.

9. A method according to claim 1 wherein said atomizer is provided with said feed stock at a feed rate of 5 ml/mn−3500 ml/mn.

10. A method according to claim 1 wherein said particles are porous.

11. A method according to claim 1 wherein said particles comprise a MMD of less than 10 microns and a MMAD of 1-5 microns.

12. A method according to claim 1 wherein said particles comprise a particle size distribution wherein at least 90% of the particles have a diameter within a range of less than 4 microns.

13. A method according to claim 1 wherein at least 90% of the droplets have a diameter within ±25% of the median droplet diameter.

14. A method according to claim 1 wherein at least 95% of the mass of the droplets have a diameter within ±25% of the median droplet diameter.

15. A method according to any one of claims 1, 13, or 14 wherein the droplets have a diameter is within ±15% of the median droplet diameter.

16. A method according to claim 15 wherein the droplets have a diameter within ±8% of the median droplet diameter.

17. A method according to claim 1 wherein said solvent is removed by heating said droplets in a gas stream to produce dried particles.

18. A method according to claim 17 wherein said dried particles are collected.

19. A method for spray drying a feed stock containing a pharmaceutical agent to produce particles suitable for pulmonary administration having a narrow particle size distribution comprising:
providing a feed stock comprising a pharmaceutically active agent at a flow rate of at least 5 ml/min;
forcing said feed stock into a manifold defined between a vibratable element and a plate and forcing the feed stock through the plate, said plate comprising holes of at least one predetermined diameter, in order to produce droplets;
drying said droplets in a gas stream to produce dried particles comprising a particle size distribution wherein at least 70% of the mass of the particles have a diameter within a 4 micron range; and
collecting said dried particles.

20. A method according to claim 19 wherein the dried particles comprise a particle size distribution wherein at least 80% of the mass of the particles have a diameter within a 4 micron range.

21. A method according to claim 19 wherein the dried particles comprise a particle size distribution wherein at least 90% of the mass of the particles have a diameter within a 4 micron range.

22. A method according to any one of claims 19-21 wherein the dried particles have a diameter within a 3 micron range.

23. A method according to any one of claims 19-21 wherein the dried particles have a diameter within a 1.5 micron range.

24. A method according to claim 19 further comprising vibrating said vibratable element in order to force said feed stock through the plate and produce droplets.

25. A method according to claim 24 wherein said plate is vibrated by coupling a piezoelectric element to said plate.

26. A method according to claim 19 wherein said holes comprise a predetermined diameter of less than 30 microns.

27. A method according to claim 19 wherein said plate comprises holes having a first diameter of less than 30 microns and a second series of holes having a second diameter of ±50% of said first diameter.

28. A method according to claim 27 wherein said second diameter is within ±20% of said first diameter.

29. A method according to claim 28 wherein said first diameter is less than 10 microns.

30. A method according to claim 19 wherein said particles are porous.

31. A method according to claim 19 wherein said particles comprise a MMD less than 10 microns and a MMAD 1-5 microns.
